(19) United States
(12) Patent Application Publication
(10) Pub. No.: US 2022/0401490 A1
(43) Pub. Date: Dec. 22, 2022

(54) METHODS FOR IMPROVING MYELOID BRIDGING IN CORD BLOOD TRANSPLANT RECIPIENTS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventor: Juliet N. BARKER, New York, NY (US)

(21) Appl. No.: 17/841,089

(22) Filed: Jun. 15, 2022

Related U.S. Application Data
(60) Provisional application No. 63/211,624, filed on Jun. 17, 2021.

(51) Int. Cl. A61K 35/28; A61K 35/51; A61P 35/02; A61P 7/06

(52) U.S. Cl. CPC A61K 35/28; A61K 35/51; A61P 35/02; A61P 7/06; A61K 2035/124

(57) ABSTRACT

The present disclosure provides methods for treating hematologic malignancies in a recipient subject in need thereof comprising administering to the recipient subject an effective amount of donor myeloid progenitor cells, and an effective amount of donor umbilical cord blood (UCB) cells, wherein the UCB cells and the myeloid progenitor cells are HLA matched. In some embodiments, the donor for the myeloid progenitor cells is not related to the recipient subject and/or the donor for the UCB cells. Also disclosed herein are methods for promoting early myeloid recovery in a recipient subject following UCB transplantation.

### CROSS-REFERENCE TO RELATED APPLICATIONS

This application claims the benefit of and priority to U.S. Provisional Patent Application No. 63/211,624 filed Jun. 17, 2021, the entire contents of which are incorporated herein by reference.

### STATEMENT OF GOVERNMENT SUPPORT

This invention was made with government support under grant number CA008748awarded by National Institutes of Health. The government has certain rights in the invention.

### TECHNICAL FIELD

The present technology relates generally to methods for treating hematologic malignancies in a recipient subject in need thereof comprising administering to the recipient subject an effective amount of donor myeloid progenitor cells, and an effective amount of donor umbilical cord blood (UCB) cells, wherein the UCB cells and the myeloid progenitor cells are HLA matched. In some embodiments, the donor for the myeloid progenitor cells is not related to the recipient subject and/or the donor for the UCB cells. Also disclosed herein are methods for promoting early myeloid recovery in a recipient subject following UCB transplantation.

### BACKGROUND

The following description of the background of the present technology is provided simply as an aid in understanding the present technology and is not admitted to describe or constitute prior art to the present technology.

Patients with otherwise incurable cancers of the blood and bone marrow frequently require life-saving allogeneic hematopoietic stem cell transplantation (HSCT). This therapy involves transplantation of blood-forming stem cells from a donor. However, many patients, especially patients from racial and ethnic minorities, will not have a suitable human leukocyte antigen (HLA) matched adult stem cell donor. An alternative stem cell source is unrelated donor cord blood (CB) collected from healthy newborn babies. CB transplants (CBT) are a routine alternative therapy approach for patients without matched adult donors and are performed to treat otherwise incurable leukemias and other cancers of the blood and bone marrow at many transplant centers around the world.

Safety and efficacy may be compromised when transplant with a large number of CB cells, e.g., greater than one unit, is required. Moreover, the risk of graft failure and GVHD is present, particularly when HLA-matching is incomplete. Another disadvantage of CBT versus bone marrow or peripheral blood HSCT is delayed recovery of the patient's donor-derived myeloid lineages early after transplant. Accordingly, there is an urgent need for developing effective CBT methods that facilitate early myeloid recovery post-transplant.

### SUMMARY OF THE PRESENT TECHNOLOGY

In one aspect, the present disclosure provides a method for treating a hematologic malignancy in a subject in need thereof comprising administering to the subject an effective amount of non-autologous myeloid progenitor cells, and an effective amount of non-autologous umbilical cord blood (UCB) cells, wherein the non-autologous UCB cells and the non-autologous myeloid progenitor cells are HLA matched and wherein the non-autologous myeloid progenitor cells and the non-autologous UCB cells are obtained from different third party donors. Also disclosed herein is a method for enhancing early myeloid recovery in a subject that is receiving umbilical cord blood (UCB) transplantation comprising administering to the subject an effective amount of non-autologous myeloid progenitor cells, and an effective amount of non-autologous umbilical cord blood (UCB) cells, wherein the non-autologous UCB cells and the non-autologous myeloid progenitor cells are HLA matched and wherein the non-autologous myeloid progenitor cells and the non-autologous UCB cells are obtained from different third party donors, optionally wherein the subject is suffering from a hematologic malignancy or disorder. In some embodiments, myeloid recovery comprises recovery of one or more of granulocytes, basophils, eosinophils, neutrophils, megakaryocytes, platelets, erythrocytes, monocytes, and macrophages. The non-autologous myeloid progenitor cells and/or non-autologous UCB cells may express CD34. Additionally or alternatively, in some embodiments of the methods disclosed herein, the UCB cells have been cryopreserved.

Additionally or alternatively, in some embodiments of the methods disclosed herein, the non-autologous UCB cells are administered as a single unit or multiple units. The non-autologous myeloid progenitor cells may be administered separately, sequentially, or simultaneously with non-autologous UCB cells. In certain embodiments, the non-autologous UCB cells are administered at a total nucleated cell (TNC) dose of about 1.0×10⁷/kg/unit to about 5.7×10⁷/kg/unit. Additionally or alternatively, in some embodiments of the methods disclosed herein, the non-autologous myeloid progenitor cells are administered at a dose of about 0.1×10⁵/kg/unit −3.1×10⁵/kg/unit.

In any of the preceding embodiments of the methods disclosed herein, the third party donor for the non-autologous myeloid progenitor cells is haploidentical to the subject. In other embodiments of the methods disclosed herein, the third party donor for the non-autologous myeloid progenitor cells is not related to the subject. Additionally or alternatively, in some embodiments of the methods disclosed herein, the third party donor for the non-autologous myeloid progenitor cells is not related to the third party donor for the non-autologous UCB cells.

In any and all embodiments of the methods disclosed herein, the third party donor for the non-autologous UCB cells is not related to the subject and/or is not HLA matched to the subject. Additionally or alternatively, in some embodiments of the methods disclosed herein, the third party donor for the non-autologous myeloid progenitor cells is an adult or a child. The non-autologous myeloid progenitor cells may be isolated from peripheral blood.

In any of the preceding embodiments of the methods disclosed herein, the subject has undergone myeloablation, and optionally has received cyclosporine-A/mycophenolate mofetil to prevent graft versus host disease. The subject may be an adult or a child. Additionally or alternatively, in some embodiments, the non-autologous UCB cells are administered in the absence of antithymocyte globulin (ATG). In certain embodiments, the subject is seropositive for CMV.

Examples of hematologic malignancies or disorders include, but are not limited to, myeloproliferative diseases, lymphomas, myelodysplastic syndrome, amegakaryocytic thrombocytopenia, acute lymphoblastic leukemia, acute myelogenous leukemia, sickle cell disease, beta thalassemia, severe combined immunodeficiency disease, marrow failure, anemia, severe aplastic anemia and Diamond-Blackfan anemia.

In any and all embodiments of the methods disclosed herein, the non-autologous myeloid progenitor cells and the non-autologous UCB cells are HLA matched at 3/8, 4/8, 5/8, 6/8, 7/8, or 8/8 HLA loci, wherein the HLA loci are HLA-A, HLA-B, HLA-C, and HLA-DRB1. In any and all embodiments of the methods disclosed herein, the non-autologous UCB cells and the subject are HLA matched at 4/6, 5/6, or 6/6 HLA loci, wherein the HLA loci are HLA-A, HLA-B, and HLA-DRB1.

### BRIEF DESCRIPTION OF THE DRAWINGS

FIG. 1 shows a pattern of whole blood chimerism in engrafting patients in the first year after haplo-dCBT (n=75). The contribution of the haploidentical donor (red/1), the dominant (engrafting) CB unit (blue/2), and the nondominant (non-engrafting) unit (green/3) to whole blood chimerism at each time point posttransplant is shown. While the haploidentical donors contributed to initial hematopoiesis, one CB unit predominated by day 28 in the majority of patients with the median donor chimerism being 100% the dominant CB unit by day 100 and beyond.

FIGS. 2A-2D show the probability of neutrophil recovery after haplo-dCBT by engraftment group (Groups 1-3, n=75). FIG. 2A shows that Group 1 patients (n=34) had early sustained myeloid recovery by day 14 posttransplant (median 12 days). FIG. 2B shows that Group 2 patients (n=20) had transient myeloid recovery (median neutrophil recovery 12 days) followed by a second nadir preceding sustained engraftment (median second neutrophil recovery 26.5 days). FIG. 2C shows percentage of Group 2 patients with a neutrophil count≥500/dL per posttransplant day. FIG. 2D shows that Group 3 patients (n=21) had delayed myeloid recovery (median 25 days).

FIGS. 3A-3C show chimerism patterns after haplo-dCBT in patients with an early myeloid bridge (Group 1, n=34). Whole blood chimerism analysis (FIG. 3A) revealed predominant engraftment of the haploidentical donor early posttransplant (median day 14 chimerism 95%, range 24-100) with subsequent increasing dominant CB unit chimerism. As of day 28 posttransplant, the majority of myeloid cells (FIG. 3B) were derived from the haploidentical donor, whereas T cells (FIG. 3C) were primarily derived from the dominant CB unit, with progressive increase in dominant CB unit-derived chimerism in all lineages thereafter. Red/1: haploidentical donor; blue/2: dominant (engrafting) CB unit; green/3: nondominant (non-engrafting) CB unit.

FIGS. 4A-4C show chimerism patterns after haplo-dCBT in patients with a transient myeloid bridge (Group 2, n=20). Whole blood chimerism analysis (FIG. 4A) revealed short-lived haploidentical donor engraftment (median day 14 haploidentical donor chimerism 82%, range 0-100%) followed by sustained CB-derived hematopoiesis. Both myeloid (FIG. 4B) and T cell (FIG. 4C) lineages were primarily derived from the dominant CB unit as early as day 28. Red/1: haploidentical donor; blue/2: dominant (engrafting) CB unit; green/3: nondominant (non-engrafting) CB unit.

FIGS. 5A-5C show chimerism patterns after haplo-dCBT in patients with no myeloid bridge and sustained CB engraftment (Group 3, n=21). Whole blood chimerism analysis (FIG. 5A) revealed that the majority of Group 3 patients had either no or minimal haploidentical donor engraftment (day 14 median chimerism 10%, range 0-94%) and the dominant CB unit predominated thereafter. The dominant CB unit was either the only or greatest contributor to myeloid (FIG. 5B) and T-cell (FIG. 5C) lineages at day 28 and beyond. Red/1: haploidentical donor; blue/2: dominant (engrafting) CB unit; green/3: nondominant (non-engrafting) CB unit.

FIGS. 6A-6D show exemplary lymphocyte subset recovery in engrafted haplo-dCBT recipients (n=75). Comparison of CD4+ T-cell (FIG. 6A), CD8+ T-cell (FIG. 6B), B-cell (FIG. 6C), and NK-cell (FIG. 6D) count recovery between haplo-dCBT recipients with optimal haploidentical donor-derived myeloid bridge (Group 1 patients, Blue/1) and haplo-dCBT recipients with either transient or no myeloid bridge (Group 2-3 patients, Red/2). Optimal myeloid bridging was not associated with improved lymphocyte subset recovery. (* p<0.01).

FIG. 7 shows a summary of infused CB and haploidentical graft characteristics. ^{a} A single haploidentical graft had a CD3+ cell dose above the 8×10³/kg cap due to inadequate purity of CD34+ selection. To avoid compromising the CD34+ dose, further T-cell depletion was not performed.

FIG. 8 shows engraftment patterns in haplo-dCBT recipients (n=77 evaluable patients). One patient was not evaluable for engraftment due to early Transplant-related mortality (TRM) on day 14. ^{b} Of the seven patients with donor-specific HLA antibodies (DSA) against the CB graft, one patient had graft failure. The remaining six patients had sustained CB engraftment (four Group 1 and two Group 3).

FIG. 9 shows a summary of factors associated with an optimal haploidentical donor-derived myeloid bridge after haplo-dCBT (n=75). Success of optimal myeloid bridge was evaluated only in patients who achieved sustained CB engraftment (n=75).

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

Double unit cord blood (dCB) transplantation (dCBT) is efficacious for adults with high-risk hematologic malignancies and has been associated with comparable progression-free survival to that of unrelated donor transplantation in multiple series (Brunstein C G et al, Blood. 2010; 116:4693-9; Milano F et al., N Engl J Med. 2016; 375:944-53; Ponce D M et al., Biol Blood Marrow Transplant. 2015 ;21:1985-93). While high rates of sustained donor engraftment have been demonstrated after dCBT, delayed count recovery is common. For example, myeloablated dCBT recipients engraft at a median of 24 days (Purtill D et al., Blood. 2014; 124:2905-12). Slow engraftment can increase morbidity, prolong hospitalization, and increase costs.

The present disclosure provides a method for improving early myeloid recovery in cord blood (CB) transplant recipients by co-administering a CB graft with peripheral blood-derived third-party donor CD34+ myeloid progenitor cells, wherein the donor CD34+ myeloid progenitor cells are HLA matched with the CB graft. These results were unexpected because according to prior studies, low CB chimerism early posttransplant was correlated with CB graft failure in those series (Kwon M, et al. Bone Marrow Transplant. 2014; 49:212-8; Tsai S B et al., Biol Blood Marrow Transplant. 2016; 22:1065-72). Also, a higher cell dose of donor haploidentical CD34+ myeloid progenitor cells (Tsai S B et al., Biol Blood Marrow Transplant. 2016; 22:1065-72; van Besien K et al., Leuk Lymphoma. 2017; 58:1512-4) and a better HLA-match between the donor of the haploidentical CD34+myeloid progenitor cells and the recipient (van Besien K et al., Leuk Lymphoma. 2017; 58:1512-4) have been associated with failure of CB engraftment in ATG-based haplo-CBT. See also Milano et al., Blood. 2019; 134 Suppl 1:146 (reporting that CB graft supplementation with a high dose CB-derived myeloid products did not enhance CB engraftment).

Conversely, in the present disclosure, graft characteristics such as low CB chimerism early posttransplant, higher HLA match between haploidentical myeloid progenitor cell donor and recipient, and higher haploidentical donor myeloid progenitor cell dose were actually associated with an increased likelihood of myeloid bridging and did not lead to CB graft failure. The Examples described herein demonstrate that a better HLA-match of third-party donor CD34+ myeloid progenitor cells to the unmanipulated CB unit, and a higher dose of third-party CD34+ myeloid progenitor cells improve the likelihood of early myeloid bridging.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, “allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor.

As used herein, “autologous” refers to deriving from or originating in the same subject or patient. An “autologous transplant” refers to the harvesting and reinfusion or transplant of a subject's own cells or organs.

As used herein, “chimerism,” refers to the presence in a subject of non-self DNA, e.g., the presence of DNA from cells that are unmatched or partially matched relative to the recipient subject. In some embodiments, “chimerism” refers to chimerism of the hematopoietic system. A determination of whether an individual is a full chimera, mixed chimera, or non-chimeric can be made by analyzing a hematopoietic cell sample from the graft recipient, e.g., peripheral blood, bone marrow, etc. Analysis may be done by any convenient method of typing. In some embodiments, the degree of chimerism amongst all mononuclear cells, T cells, B cells, CD56+ NK cells, and CD15+ neutrophils is regularly monitored, using PCR with probes for microsatellite analysis. For example, commercial kits that distinguish polymorphisms in short terminal repeat lengths of donor and host origin are available. Automated readers provide the percentage of donor type cells based on standard curves from artificial donor and host cell mixtures.

Individuals who exhibited more than a 95% donor cells in a given blood cell lineage by such analysis at any time post-transplantation are referred to as having full donor chimerism in this transplant patient group. Mixed chimerism is defined as greater than 1% donor but less than 95% donor DNA in such analysis. Individuals who exhibit mixed chimerism may be further classified according to the evolution of chimerism, where improving mixed chimerism is defined as a continuous increase in the proportion of donor cells over at least a 6-month period. Stable mixed chimerism is defined as fluctuations in the percentage of recipient cells over time, without complete loss of donor cells. Candidates for withdrawal of immunosuppression have mixed chimerism until at least 6 months post-transplantation.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

“Hematopoietic stem cell” or “HSC” refers to a clonogenic, self-renewing pluripotent cell capable of ultimately differentiating into all cell types of the hematopoietic system, including B cells, T cells, NK cells, lymphoid dendritic cells, myeloid dendritic cells, granulocytes, macrophages, megakaryocytes, and erythroid cells. As with other cells of the hematopoietic system, HSCs are typically defined by the presence of a characteristic set of cell markers. “Enriched” when used in the context of HSC refers to a cell population selected based on the presence of a single cell marker, generally CD34+, while “purified” in the context of HSC refers to a cell population resulting from a selection on the basis of two or more markers, preferably CD34+ CD90+.

As used herein, “major histocompatibility complex” antigens (“MHC”, also called “human leukocyte antigens”, HLA) are protein molecules expressed on the surface of cells that confer a unique antigenic identity to these cells. MHC/HLA antigens are target molecules that are recognized by T-cells and natural killer (NK) cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells. Both have been implicated in the rejection of transplanted organs.

An important aspect of the HLA gene system is its polymorphism. Each gene, MHC class I (A, B and C) and MHC class II (DP, DQ and DR) exists in different alleles. HLA alleles are designated by numbers and subscripts. For example, two unrelated individuals may carry class I HLA-B, genes B5, and Bw41, respectively. Allelic gene products differ in one or more amino acids in the c and/or P domain(s). Large panels of specific antibodies or nucleic acid reagents are used to type HLA haplotypes of individuals, using leukocytes that express class I and class II molecules. The genes most important for HLA typing are the six MHC Class I and Class II proteins, two alleles for each of HLA-A; HLA-B and HLA-DR.

The HLA genes are clustered in a “super-locus” present on chromosome position 6p21, which encodes the six classical transplantation HLA genes and at least 132 protein coding genes that have important roles in the regulation of the immune system as well as some other fundamental molecular and cellular processes. The complete locus measures roughly 3.6 Mb, with at least 224 gene loci. One effect of this clustering is that “haplotypes,” i.e. the set of alleles present on a single chromosome that is inherited from one parent, tend to be inherited as a group. The set of alleles inherited from each parent forms a haplotype, in which some alleles tend to be associated together. Identifying a patient's haplotypes can help predict the probability of finding matching donors and assist in developing a search strategy, because some alleles and haplotypes are more common than others and they are distributed at different frequencies in different racial and ethnic groups. An HLA “haploidentical” donor is one who shares, by common inheritance, exactly one HLA haplotype with the recipient and is mismatched for a variable number of HLA genes, ranging from zero to six, on the unshared haplotype. Potential HLA-haploidentical donors include biological parents; biological children; full or half siblings; and even extended family donors such as aunts, uncles, nieces, nephews, cousins, or grandchildren. In some cases, one haplotype of the donor is matched to the recipient whereas the other haplotype is mismatched. This situation is frequently found with organs from living or deceased donors. HLA mismatched donor/recipient pairs have an increased risk of GVHD relative to perfectly matched pairs (i.e. where all 6 alleles are matched).

As used herein, the term “HLA matched” in the context of UCB and/or myeloid progenitor cells refers to a situation where there is matching at 4/6, 5/6 or 6/6 HLA loci (e.g., HLA-A, HLA-B antigen, and DRB1 allele) or in particular embodiments with respect to UCB and/or myeloid progenitor cells, e.g., from an adult source, refers to situations where there is matching at 3/8, 4/8, 5/8, 6/8, 7/8, or 8/8 HLA loci (e.g., HLA-A, HLA-B, HLA-C antigen, and DRB1 allele). . Accordingly, the term “HLA matched” encompasses both partial (4/6, 5/6, 3/8, 4/8, 5/8, 6/8, 7/8) and complete (6/6, 8/8) matching at HLA loci (e.g., HLA-A, HLA-B, HLA-C antigen, and DRB1 allele).

Also, unless otherwise noted, “unmatched,” or “not HLA matched,” as used herein in the context of UCB and/or myeloid progenitor cells, refers to matching at 0/6, 1/6, 2/6 or 3/6 HLA loci, or in particular embodiments with respect to UCB and/or myeloid progenitor cells, e.g., from an adult source, refers to matching at 0/8, 1/8 or 2/8 HLA loci. “HLA matched,” and “not HLA matched” can, for example, refer to the relationship between the donor UCB cells, and donor myeloid progenitor cells, between units of donor UCB cells, and/or between the donor UCB cells, and/or donor myeloid progenitor cells and the subject that is the recipient of the donor cells.

As used herein, a “recipient” is an individual to whom an organ, tissue or cells from another individual (“donor”), commonly of the same species, has been transferred. For the purposes of the present disclosure, a recipient and a donor are either HLA matched or HLA mismatched.

As used herein, the term “related” in the context of UCB cells or myeloid progenitor cells, refers to self, or to a first or second degree blood relative. For example, UCB that is related to the subject refers to UCB from the subject itself, or from a first or second degree blood relative of the subject. In another example, UCB that is related to myeloid progenitor cells refers to UCB and myeloid progenitor cells that are from the same donor, or donors that are first or second degree blood relatives. Likewise, unless otherwise noted, “unrelated,” in these contexts, refers to relationships that are more distant than that of a second degree blood relative.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

A “unit,” as used herein (e.g., in the context of transplantation), of UCB or cells therefrom, refers to UCB or cells therefrom from a single umbilical cord. In certain embodiments, such methods comprise administering one unit of UCB, or cells therefrom. In another embodiment, the methods presented herein comprise administering multiple units of UCB, or cells therefrom. For example, the methods presented herein can comprise administering one, two, three, or four units of UCB, or cells therefrom. In instances wherein greater than one unit of UCB cells is used, in certain embodiments, at least a portion or all of the UCB cells can be unrelated to the subject, to the myeloid progenitor cells, and/or to other portions of the UCB cells (e.g., other UCB cell units). In instances wherein greater than one unit of UCB cells is used, in certain embodiments, at least a portion of the UCB cells, can be unmatched or partially matched to the subject, and/or to other portions of the UCB cell units. In another embodiment, the methods presented herein can comprise administering less than one unit of UCB, or cells therefrom. For example, the methods presented herein can comprise administering 0.2, 0.3, 0.4, 0.5, 0.6, 0.7, 0.8, or 0.9 units of UCB, or cells therefrom. In particular embodiments, the methods presented herein can comprise administering a particular number of units (less than one, one, or more than one) over multiple administrations.

### Umbilical Cord Blood Cells

Umbilical cord blood (also referred to herein as UCB or “cord blood”) for use in accordance with the present disclosure may be collected in any medically or pharmaceutically-acceptable manner and may be present in a composition, e.g., a pharmaceutical composition. Various methods for the collection of cord blood have been described. See, e.g., U.S. Pat. Nos. 6,102,871; 6,179,819; and 7,147,626, the contents of each of which are incorporated by reference in its entirety. A conventional technique for the collection of cord blood is based on the use of a needle or cannula, which is used with the aid of gravity. Cord blood may be collected into, for example, blood bags, transfer bags, or sterile plastic tubes.

In some embodiments, non-autologous umbilical cord blood is obtained from a commercial cord blood bank (e.g., LifeBankUSA, etc.). In another embodiments, non-autologous umbilical cord blood is collected from a post-partum mammalian umbilical cord and used immediately (e.g., within 1, 2, 3, 4, 5, 6, 7, 8, 9, 10, 11, or 12 hours of collection). In other embodiments, the non-autologous cord blood used to treat a subject is cord blood that has been cryopreserved. Non-autologous umbilical cord blood can be collected from a single umbilical cord or from a plurality of umbilical cords.

In certain embodiments, the non-autologous UCB cells are unrelated to the subject and/or the non-autologous myeloid progenitor cells. In another embodiment, the non-autologous UCB cells, are matched to the subject and/or the non-autologous myeloid progenitor cells. In yet another embodiment, the non-autologous UCB cells are not HLA matched to the non-autologous myeloid progenitor cells. In still another embodiment, the non-autologous UCB cells are unrelated and unmatched to the non-autologous myeloid progenitor cells. In particular embodiments the non-autologous UCB is matched to the subject at 3/6, 4/6, or 5/6 HLA loci. In particular embodiments the non-autologous UCB cells, e.g., from an adult source, are matched to the subject at 6/8, 7/8, or 8/8 HLA loci.

In some embodiments, non-autologous umbilical cord blood is prepared from preterm umbilical cord. In other embodiments, non-autologous umbilical cord blood is prepared from full-term umbilical cord. In certain embodiments, non-autologous umbilical cord blood is obtained from a post-partum mammalian umbilical cord of a full-term birth. In other embodiments, non-autologous umbilical cord blood is obtained from a post-partum mammalian umbilical cord of a premature birth. In some embodiments, the umbilical cord is the umbilical cord of an infant born at about 23 to about 25 weeks of gestation. In some embodiments, the umbilical cord is the umbilical cord of an infant born at about 26 to about 29 weeks of gestation. In some embodiments, the umbilical cord is the umbilical cord of an infant born at about 30 to about 33 weeks of gestation. In some embodiments, the umbilical cord is the umbilical cord of an infant born at about 34 to about 37 weeks of gestation. In some embodiments, the umbilical cord is the umbilical cord of an infant born at about 37 to about 42 weeks of gestation.

Non-autologous cord blood, or cells obtained therefrom (e.g., total nucleated cells or stem cells derived therefrom), may be collected from a single individual (i.e., as a single unit) for administration, or may be pooled with other units. In certain embodiments, the non-autologous cord blood, or cells obtained therefrom (e.g., total nucleated cells or stem cells derived therefrom) is stored prior to use. Where non-autologous umbilical cord blood is pooled from a plurality of umbilical cords, the pooled cord blood can comprise umbilical cord blood from full-term births only, cord blood from a combination of full-term births and premature births, or cord blood from premature births only. For example, non-autologous cord blood from the umbilical cord of a premature infant can be combined with, e.g., cord blood from other premature infants, cord blood from full-term births only, or a combination of cord blood from both premature and full-term births. Non-autologous cord blood can also be combined with peripheral blood. In certain embodiments, non-autologous cord blood from premature births is used, as such cord blood comprises relatively high numbers of CD34+ stem cells per unit volume, compared to cord blood from full-term births.

In certain embodiments, a unit of non-autologous cord blood contains a sufficient number of cells such that at least about 1.0×10⁶, 1.5×10⁶, 2.0×10⁶, 1.5×10⁶, 2.0×10⁶, 2.5×10⁶, 3.0×10⁶, 3.5×10⁶, 4.0×10⁶, 4.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ cells obtained from said cord blood, e.g., total nucleated cells from cord blood, per kilogram body weight of a subject are administered. In certain embodiments, one unit of non-autologous cord blood or cells obtained therefrom is administered. In certain embodiments, less than one unit is administered. In certain embodiments, more than one unit is administered, e.g., two or more (e.g., 2, 3, 4, 5, 6, or more) units are administered.

### Myeloid Progenitor Cells

Non-autologous myeloid progenitor cells, e.g., human myeloid progenitor cells, for use in accordance with the present disclosure may be collected in any medically or pharmaceutically-acceptable manner and may be present in a composition, e.g., a pharmaceutical composition. In certain embodiments, a composition (e.g., a pharmaceutical composition, i.e., a pharmaceutical grade solution suitable for administration to a human) provided herein comprises non-autologous myeloid progenitor cells. In certain embodiments, the composition comprises non-autologous myeloid progenitor cells obtained from peripheral blood.

In one embodiment, the non-autologous myeloid progenitor cells are sterile. In one embodiment, the population of non-autologous myeloid progenitor cells is heterogeneous. In certain embodiments, the non-autologous myeloid progenitor cells are obtained from a single donor. In certain embodiments, the population of non-autologous myeloid progenitor cells are obtained from more than one donor. In embodiments wherein the non-autologous myeloid progenitor cells are obtained from more than one donor, the cells from the different donors need not be related to each other. Non-autologous myeloid progenitor cells for use in accordance with the present disclosure are generally unrelated to the subject recipient of the cells. Non-autologous myeloid progenitor cells for use in accordance with the present disclosure may be unmatched or HLA matched to the subject recipient of the cells.

### Collection of UCB and Myeloid Progenitor Cells

Umbilical cord blood cells, e.g., UCB, and myeloid progenitor cells, can be obtained using methods known in the art and according to procedures established by medical practitioners. In one embodiment, the umbilical cord or umbilical cord blood and/or peripheral blood is recovered from a donor by informed consent and a complete medical history of the donor is also taken. These medical records can be used to coordinate subsequent use of the peripheral blood or UCB, for example, UCB cells or myeloid progenitor cells harvested therefrom.

In certain embodiments, the cord blood is recovered. In specific embodiments, the umbilical cord is subjected to a conventional cord blood recovery process. Cord blood may also be obtained from a commercial cord blood banking service, e.g., LifeBankUSA, Cedar Knolls, N.J. In certain embodiments, umbilical cord blood is collected using an umbilical cord blood collection kit such as described in U.S. Pat. No. 7,147,626, the contents of which are incorporated by reference in their entirety.

In one embodiment, collection kits, containing standard chucks, sterile gauze pad, povidine iodine swabs, sterile alcohol pads, plastic umbilical cord blood clamps, slide clip or hemostat clamps and leak proof resealable bags or canisters are used. The collection can be performed before the placenta is delivered (in utero collection), after the placenta is delivered (ex utero collection) or during a Caesarian section, prior to delivery of placenta. Briefly, the venipuncture site on the distal site on the umbilical cord is sterilized. The collection tubing leading from the large collection bag is clamped, the cap is removed from the needle, and the umbilical vein is cannulated with the bevel of the needle facing down toward the umbilical vein. The clamp is removed to allow the blood to flow and collection bag is lowered below the cannulation site to allow the blood to fill the collection bag by gravity. When the blood flow stops, the venipuncture site is clamped and the needle is withdrawn from the umbilical vein. The collection bag is labeled and put into the insulated shipping container. The placenta with the clamped umbilical cord blood is placed in the leak proof resealable bag and the bag is then properly sealed and labeled. After collection, viability of umbilical cord blood cells is determined by hemocytometer after trypan blue staining.

In certain embodiments, the proximal umbilical cord is clamped, e.g., within 3-4 inches of the insertion into the placental disc prior to cord blood recovery. In other embodiments, the proximal umbilical cord is clamped after cord blood recovery. Conventional techniques for the collection of cord blood may be used. In one embodiment, a needle or cannula is used, with the aid of gravity, to drain cord blood.

In specific embodiments, myeloid progenitor cells are isolated from peripheral blood using techniques known by those skilled in the art, such as, for example, density gradient centrifugation. In one embodiment, myeloid progenitor cells are isolated by differential centrifugation in order to separate the cells from, e.g., cell debris, serum, etc. In a specific embodiment, myeloid progenitor cells can be recovered from peripheral blood by centrifugation at, e.g., about 5000×g for about 15 minutes at room temperature, which separates cells from contaminating debris and platelets. The cell pellets are resuspended in, e.g., IMDM serum-free medium containing 2 U/ml heparin and 2 mM EDTA (GibcoBRL, NY). The myeloid progenitor cells can be isolated using leukapheresis, e.g., using a commercial collection kit such as LYMPHOPREP™ (Nycomed Pharma, Oslo, Norway). Cells may then counted using, e.g., a hemocytometer. Viability is typically evaluated by trypan blue exclusion.

In other embodiments, the cells collected from the UCB or peripheral blood are cryopreserved for use at a later time. Methods for cryopreservation of cells, such as stem cells, are well known in the art, for example, cryopreservation using the methods of Boyse et al. (U.S. Pat. No. 5,192,553, issued Mar. 9, 1993) or Hu et al. (WO 00/73421, published Dec. 7, 2000).

### Therapeutic Methods of the Present Technology

In one aspect, provided herein are methods of transplanting non-autologous UCB cells to a subject, e.g., a human subject, comprising administering to the subject an effective amount of non-autologous myeloid progenitor cells, and an effective amount of non-autologous umbilical cord blood (CB) cells. Sources of non-autologous myeloid progenitor cells that can be used in the methods described herein include, for example, bone marrow or cells therefrom, or peripheral blood or cells therefrom.

In one embodiment, provided herein is a method of transplanting non-autologous human umbilical cord blood cells (UCB) cells, e.g., human umbilical cord blood, to a subject, e.g., a human subject, comprising administering the non-autologous human umbilical cord blood cells (UCB) cells, e.g., human umbilical cord blood, in combination with non-autologous myeloid progenitor cells e.g., human myeloid progenitor cells. In one embodiment, the non-autologous human UCB cells, e.g., human UCB, are not related to the subject. In a particular embodiment, the non-autologous UCB cells, e.g., human UCB, are HLA matched to the subject.

In another embodiment, the non-autologous myeloid progenitor cells are not related to the subject. In a particular embodiment, the non-autologous myeloid progenitor cells are haploidentical to the subject. In another particular embodiment, the non-autologous myeloid progenitor cells are not HLA matched to the subject. In yet another embodiment, the non-autologous human UCB cells, e.g., human UCB, are unrelated to the subject and the non-autologous myeloid progenitor cells are unrelated to the subject. In still another embodiment, the non-autologous UCB cells, e.g., UCB, are unrelated and matched to the subject and the non-autologous myeloid progenitor cells are unrelated and HLA matched or not HLA matched to the subject. In one embodiment, non-autologous myeloid progenitor cells are unrelated and HLA matched to the non-autologous UCB cells, e.g., UCB. In one embodiment, non-autologous myeloid progenitor cells are (a) unrelated and HLA matched to the non-autologous UCB cells, e.g., UCB, and (b) are unrelated and not HLA matched to the recipient.

In another aspect, provided herein are methods for inducing chimerism in a subject, comprising administering to the subject a combination of non-autologous UCB cells, e.g., UCB, and non-autologous myeloid progenitor cells, wherein at least a portion of the non-autologous UCB cells are HLA matched to the subject, and/or the non-autologous myeloid progenitor cells are not HLA matched or HLA matched to the subject, such that chimerism in the subject occurs.

In one embodiment of such methods, greater than one unit of non-autologous UCB cells is administered to the subject, e.g., 2, 3, or 4 units of non-autologous UCB cells are administered to the subject. In particular embodiments wherein greater than one unit of non-autologous UCB cells is administered to the subject the method of inducing chimerism can result in multiple chimerism, that is, chimerism involving greater than one, and up to all, of the administered non-autologous UCB cell, units, or progeny thereof, can result.

In another embodiment of such methods, chimerism involving the non-autologous myeloid progenitor cells or progeny thereof can result. In yet another embodiment, chimerism involving the non-autologous UCB cells (including multiple chimerism in instances wherein greater than one unit of non-autologous UCB cells, is administered), or progeny thereof, and the non-autologous myeloid progenitor cells, or progeny thereof, can result.

In still yet another embodiment of such methods, the non-autologous UCB cells are unrelated to the subject. In instances in which greater than one unit of non-autologous UCB is administered, one or more of the non-autologous UCB cell units can be unrelated to the subject.

In a particular embodiment of such methods, the non-autologous myeloid progenitor cells are unrelated to the subject and can, additionally, be unrelated to the non-autologous UCB cells. In still another embodiment of such methods, both the non-autologous UCB cells, and the non-autologous myeloid progenitor cells are unrelated to the subject.

In certain embodiments of such methods, chimerism (comprising non-autologous UCB cells or progeny thereof, and/or non-autologous myeloid progenitor cells or progeny thereof) is first detected in the subject within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 days, or more of administration of the non-autologous UCB cells, in combination with the non-autologous myeloid progenitor cells to the subject.

Chimerism can be detected using methods known in the art. For example, chimerism can be detected using blood samples. In one embodiment, chimerism is detected using a polymerase chain reaction (PCR)-based method, e.g., by short tandem repeat assays. In one embodiment, a test for chimerism after UBC transplantation involves identifying the genetic profiles of the recipient and of the donor and then evaluating the extent of mixture in the recipient's blood, bone marrow, or other tissue. Chimerism testing (engraftment analysis) by DNA employs methodology commonly used in human identity testing and is accomplished by the analysis of genomic polymorphisms called short tandem repeat (STR) loci. In one embodiment, quantitation (e.g., using short tandem repeat assays) of peripheral blood donor chimerism is assessed on Days 7, 14, 30, 60, 100 and 180 (+/−10 days), with quantitation (e.g., using short tandem repeat assays) of peripheral blood recipient chimerism assessed at baseline along with chimerism of the donor cells (UCB and myeloid progenitor cells) at baseline.

In still another aspect, provided herein are methods for cell engraftment, e.g., platelet or neutrophil engraftment, in a subject, comprising administering to the subject a combination of non-autologous UCB cells, e.g., UCB, and non-autologous myeloid progenitor cells, wherein at least a portion of the non-autologous UCB cells are HLA matched to the subject, and/or the non-autologous myeloid progenitor cells are not HLA matched or HLA matched to the subject, such that cell engraftment in the subject occurs. In certain embodiments, the cell engraftment comprises engraftment of non-autologous UCB cells or progeny thereof. In certain other embodiments, the cell engraftment comprises engraftment of non-autologous myeloid progenitor cells or progeny thereof. In still other embodiments, the engraftment comprises engraftment of non-autologous UCB cells or progeny thereof, and non-autologous myeloid progenitor cells or progeny thereof. In certain embodiments, a method of cell engraftment provided herein shortens the time to engraftment.

In one embodiment of such methods, the non-autologous UCB cells are unrelated to the subject. In a particular embodiment, the non-autologous UCB cells are HLA matched to the subject. In another particular embodiment, the non-autologous myeloid progenitor cells are unrelated to the subject and can, additionally, be unrelated to the non-autologous UCB cells. In a particular embodiment, the non-autologous myeloid progenitor cells are HLA matched to the subject. In another particular embodiment, the non-autologous myeloid progenitor cells are not HLA matched to the subject. In yet another embodiment, the non-autologous UCB cells are unrelated to the subject and the non-autologous myeloid progenitor cells are unrelated to the subject. In still another embodiment, the non-autologous UCB cells are unrelated and HLA matched to the subject and the non-autologous myeloid progenitor cells are unrelated and HLA matched or not HLA matched to the subject. In certain embodiments, the methods presented herein exhibit an enhanced ability to engraft as compared to administration of non-autologous UCB cells, alone.

Engraftment can be detected using methods known in the art. For example, in one embodiment, a complete blood count with differential may be performed every 1-3 days from Day 0 to until absolute neutrophil count>500/mm³ for 3 days after nadir is reached and until platelet count reaches ≥20,000/mm³ for 3 consecutive measurements on 3 different days and independence from platelet transfusion for a minimum of 7 days. As used herein, “neutrophil engraftment” refers to the first of three days following the neutrophil nadir with an absolute neutrophil count above 500/mm³. As used herein, “platelet engraftment” refers to the first of three consecutive days demonstrating a platelet count ≥20,000/mm³, after a seven day period of platelets ≥20,000/mm³ without transfusions.

In certain embodiments, cell engraftment in the subject is detected within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 days, or 2 months, 2.5 months, 3 months, or more of administration of the non-autologous UCB cells, in combination with non-autologous myeloid progenitor cells to the subject.

In certain embodiments, the methods presented herein comprise administering one unit of non-autologous UCB cells, e.g., UCB. In another embodiment, the methods presented herein comprise administering multiple units of non-autologous UCB cells, e.g., UCB. For example, the methods presented herein can comprise administering two, three, or four units of non-autologous UCB cells, e.g., UCB.

In certain embodiments, the methods presented herein comprise administering non-autologous UCB cells, e.g., UCB, concurrently with the non-autologous myeloid progenitor cells. In a particular embodiment, the non-autologous UCB cells are administered to a subject simultaneously. In another embodiment, the non-autologous UCB cells and non-autologous myeloid progenitor cells are administered to the subject within 0.5, 1, 1.5, 2, 3, 4, 6, 8, 10, 12, 16, 18, or 24 hours or more, or within 1, 2, 3, 4, 5, 6, or 7 days or more of each other. In a specific embodiment, the non-autologous UCB cells, e.g., UCB, is administered to the subject, then the myeloid progenitor cells, is administered, e.g., is administered within 1 hour of administration of UCB, or within the minimum period necessary to verify that the subject is not exhibiting an adverse reaction to the UCB administration.

The methods provided herein can exhibit advantages that can include, for example, a reduction in the length of time to cell engraftment, limiting the time the subject is neutropenic, limiting the time the subject is thrombocytopenic, and establishment of chimerism, relative to administration of non-autologous UCB cells, e.g., UCB, alone.

The ratio of non-autologous UCB cells, and non-autologous myeloid progenitor cells administered can vary. The ratio of non-autologous UCB cells, and non-autologous myeloid progenitor cells can be determined according to the judgment of those of skill in the art. In certain embodiments, the ratio of non-autologous UCB cells, to non-autologous myeloid progenitor cells is about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000. In certain embodiments, the ratio of non-autologous UCB cells, to non-autologous myeloid progenitor cells is between about 20:1 and about 1:20, or is about 1:10, about 1:5, about 1:1, about 5:1 or about 10:1.

Administration of non-autologous UCB cells, and non-autologous myeloid progenitor cells can be performed using any technique for cell administration known in the art. In one embodiment, administration is venous, for example, intravenous, e.g., through an IV, PICC line, central line, etc. For example, non-autologous UCB cells, and non-autologous myeloid progenitor cells may be administered, in separate compositions or in a single composition, to a subject in any pharmaceutically or medically acceptable manner, including by injection or transfusion. In certain embodiments, the composition(s) may be formulated as an injectable composition (e.g., WO 96/39101, incorporated herein by reference in its entirety).

In certain embodiments, non-autologous UCB cells, or non-autologous myeloid progenitor cells are administered to a subject parenterally. The term “parenteral” as used herein includes subcutaneous injections, intravenous, intramuscular, intra-arterial injection, or infusion techniques. In certain embodiments, non-autologous UCB cells, or non-autologous myeloid progenitor cells are administered to a subject intravenously. In certain other embodiments, non-autologous UCB cells, or non-autologous myeloid progenitor cells are administered to a subject intraventricularly.

Non-autologous UCB cells, and non-autologous myeloid progenitor cells may be contained, separately or together, in any pharmaceutically-acceptable carrier. For example, non-autologous UCB cells, or non-autologous myeloid progenitor cells may be carried, stored, or transported in any pharmaceutically or medically acceptable container, for example, a blood bag, transfer bag, plastic tube, syringe, vial, or the like.

Administration of non-autologous UCB cells, and/or non-autologous myeloid progenitor cells to a subject can be performed once or a plurality of times. In certain embodiments, administration is performed once. In certain embodiments, administration is performed a plurality of times, e.g., two, three, four, or more times. In certain embodiments, non-autologous UCB cells, are administered a plurality of times. In certain embodiments, non-autologous myeloid progenitor cells are administered a plurality of times.

In certain embodiments, the amount of non-autologous cord blood or cells obtained therefrom (e.g., total nucleated cells from umbilical cord blood) administered to a subject in accordance with the methods described herein can be determined based on the number of cells present in the cord blood. The amount or number of non-autologous UCB or cells obtained therefrom (e.g., total nucleated cells from umbilical cord blood) administered to the subject depends on the source of umbilical cord blood or cells obtained therefrom (e.g., total nucleated cells from umbilical cord blood), the severity or nature of disorders or conditions to be treated, as well as age, body weight and physical condition of the subject, etc. In certain embodiments, about 0.01 to about 0.1, about 0.1 to about 1, about 1 to about 10, about 10 to about 10², about 10² to about 10³, about 10³ to about 10⁴, about 10⁴ to about 10⁵, about 10⁵ to about 10⁶, about 10⁶ to about 10⁷, about 10⁷ to about 10⁸, or about 10⁸ to about 10⁹ non-autologous umbilical cord blood cells (e.g., total nucleated cells from umbilical cord blood), or total non-autologous umbilical cord blood cells per kilogram body weight of a subject are administered. In various embodiments, at least about 0.1, 1, 10, 10², 10³, 10⁴, 10⁵, 10⁶, 10⁷, 10⁸, or 10⁹ non-autologous umbilical cord blood cells (e.g., total nucleated cells from umbilical cord blood), or non-autologous umbilical cord blood cells per kilogram body weight of a subject are administered.

In specific embodiments, at least about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ non-autologous umbilical cord blood cells (e.g., total nucleated cells from umbilical cord blood), non-autologous myeloid progenitor cells, non-autologous umbilical cord blood cells per kilogram body weight and/or non-autologous myeloid progenitor cells per kilogram body weight of a subject are administered.

In a more specific embodiment, at least about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, or 5.0×10⁶ non-autologous myeloid progenitor cells per kilogram body weight of a subject are administered. In a more specific embodiment, at least about 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ non-autologous umbilical cord blood cells (e.g., total nucleated cells from umbilical cord blood) per kilogram body weight of a subject are administered.

In various embodiments, at most about 10⁴, 10⁵, 10⁶, 10⁷, 10⁸, or 10⁹ non-autologous umbilical cord blood cells, non-autologous myeloid progenitor cells, non-autologous umbilical cord blood cells per kilogram body weight, or non-autologous myeloid progenitor cells per kilogram body weight of a subject are administered. In specific embodiments, at most about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ non-autologous umbilical cord blood cells (e.g., total nucleated cells from umbilical cord blood), non-autologous myeloid progenitor cells, non-autologous umbilical cord blood cells per kilogram body weight, or non-autologous myeloid progenitor cells per kilogram body weight of a subject are administered.

In a more specific embodiment, at most about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, or 5.0×10⁶ non-autologous myeloid progenitor cells per kilogram body weight of a subject are administered. In a more specific embodiment, at most about 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ non-autologous umbilical cord blood cells (e.g., total nucleated cells from umbilical cord blood) per kilogram body weight of a subject are administered. In specific embodiments, a greater number of non-autologous umbilical cord blood cells (e.g., total nucleated cells from umbilical cord blood) than non-autologous myeloid progenitor cells per kilogram body weight of a subject are administered.

In certain embodiments, at least about 10⁴ to about 10⁷, for example, 0.5×10⁴, 1.0×10⁴, 1.5×10⁴, 2.0×10⁴, 2.5×10⁴, 3.0×10⁴, 3.5×10⁴, 4.0×10⁴, 4.5×10⁴, 5.0×10⁴, 5.5×10⁴, 6.0×10⁴, 6.5×10⁴, 7.0×10⁴, 7.5×10⁴, 8.0×10⁴, 8.5×10⁴, 9.0×10⁴, 9.5×10⁴, 0.5×10⁵, 1.0×10⁵, 1.5×10⁵, 2.0×10⁵, 2.5×10⁵, 3.0×10⁵, 3.5×10⁵, 4.0×10⁵, 4.5×10⁵, 5.0×10⁵, 5.5×10⁵, 6.0×10⁵, 6.5×10⁵, 7.0×10⁵, 7.5×10⁵, 8.0×10⁵, 8.5×10⁵, 9.0×10⁵, 9.5×10⁵, 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷, non-autologous CD34+ cells per kilogram body weight are administered. Such CD34+ cells can be from cord blood and/or peripheral blood.

In certain embodiments, when the non-autologous CD34+cells are from cord blood and peripheral blood, the percentage of non-autologous CD34+ myeloid progenitor cells relative to total non-autologous myeloid progenitor cells is greater than the percentage of non-autologous

CD34+ UCB cells relative to total non-autologous UCB cells. In one embodiment, the proportion of CD34+ cells in the myeloid progenitor cell population is 0.1-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-65, 6.5-7.0, 7.0-7.5, 7.5-8.0, 8.0-8.5, 8.5-9.0, 9.0-9.5, or 9.5-10.0 fold or higher compared to the proportion of CD34+ cells in the cord blood cell population. In certain embodiments, 0.1%, 0.5%, 1.0%, 1.5%, 2.0%, 2.5%, 3.0%, 3.5%, 4.0%, 4.5%, or 5.0% or greater of the myeloid progenitor cells are CD34+.

The non-autologous UCB cells, e.g., UCB, and non-autologous myeloid progenitor cells, can be delivered in a volume appropriate for the size of the subject. Typical blood volume of a human adult is about 85-100 mL/kg body weight. Thus, the blood volume for human adults ranges from approximately 40 mL to approximately 300 mL. In various embodiments, non-autologous UCB cells, e.g., UCB, and/or non-autologous myeloid progenitor cells are administered in a total volume of about 0.5 mL, 1.0 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, or about 30 mL, or more. The administration of such volumes can be a single administration or in multiple administrations. The time over which such volumes of non-autologous cord blood or number of non-autologous cord blood cells, or non-autologous myeloid progenitor cells can be administered can vary from, e.g., 0.5 hours, 1 hour, 1.5 hours, 2 hours, 2.5 hours, 3 hours, 3.5 hours, 4 hours, or more. In certain embodiments, small transfusions under 20 mL are performed using a syringe. Larger-volume transfusions can administered by an infusion device, e.g., within a period of one to four hours.

The methods provided herein can be performed on any subject in need thereof. In one aspect, the subject is in need of hematopoietic reconstitution, partial reconstitution, or augmentation. In certain embodiments, the subject is a human subject. In certain embodiments, the subject is an adult human subject. In certain embodiments, the subject is 25 years or younger. In certain embodiments, the subject is a child or an infant.

In certain embodiments, prior to the methods presented herein, e.g., methods of transplanting, inducing chimerism and/or methods of engraftment, the subject has been administered one or more of myeloablative conditioning, using, e.g., TBI, Clofarabine, and/or Ara-C1; reduced toxicity conditioning (also referred to as reduced intensity conditioning) using, e.g., Busulfan, Fludarabine, and/or Alemtuzumab; radiation therapy; chemotherapy; or other therapy such as immunosuppressive therapy or a therapy that reduces blood cell count. In a particular embodiment, wherein a subject has received one or more of the above, the subject exhibits complete myeloablation. In one embodiment, at least some immune system function is retained.

In another particular aspect, the methods provided herein can be used as methods for the treatment of a hematologic disorder or malignancy, e.g., a lymphohematopoietic malignancy, myelodysplastic syndrome, amegakaryocytic thrombocytopenia, leukemias such as acute lymphoblastic leukemia (ALL) and acute myelogenous leukemia (AML), neutropenia, sickle cell disease such as sickle cell anemia, beta thalassemia (e.g. beta thalassemia major), severe combined immunodeficiency disease, marrow failure, or anemia such as severe aplastic anemia or Diamond-Blackfan anemia in a subject in need thereof.

In one aspect, the present disclosure provides a method for treating a hematologic malignancy in a subject in need thereof comprising administering to the subject an effective amount of non-autologous myeloid progenitor cells, and an effective amount of non-autologous umbilical cord blood (UCB) cells, wherein the non-autologous UCB cells and the non-autologous myeloid progenitor cells are HLA matched and wherein the non-autologous myeloid progenitor cells and the non-autologous UCB cells are obtained from different third party donors. Also disclosed herein is a method for enhancing early myeloid recovery in a subject that is receiving umbilical cord blood (UCB) transplantation comprising administering to the subject an effective amount of non-autologous myeloid progenitor cells, and an effective amount of non-autologous umbilical cord blood (UCB) cells, wherein the non-autologous UCB cells and the non-autologous myeloid progenitor cells are HLA matched and wherein the non-autologous myeloid progenitor cells and the non-autologous UCB cells are obtained from different third party donors, optionally wherein the subject is suffering from a hematologic malignancy or disorder. In some embodiments, myeloid recovery comprises recovery of one or more of granulocytes, basophils, eosinophils, neutrophils, megakaryocytes, platelets, erythrocytes, monocytes, and macrophages. The non-autologous myeloid progenitor cells and/or non-autologous UCB cells may express CD34. Additionally or alternatively, in some embodiments of the methods disclosed herein, the UCB cells have been cryopreserved.

Additionally or alternatively, in some embodiments of the methods disclosed herein, the non-autologous UCB cells are administered as a single unit or multiple units. The non-autologous myeloid progenitor cells may be administered separately, sequentially, or simultaneously with non-autologous UCB cells. In certain embodiments, the non-autologous UCB cells are administered at a total nucleated cell (TNC) dose of about 1.0×10⁷/kg/unit to about 5.7×10⁷/kg/unit. Additionally or alternatively, in some embodiments of the methods disclosed herein, the non-autologous myeloid progenitor cells are administered at a dose of about 0.1×10⁵/kg/unit −3.1×10⁵/kg/unit.

In any of the preceding embodiments of the methods disclosed herein, the third party donor for the non-autologous myeloid progenitor cells is haploidentical to the subject. In other embodiments of the methods disclosed herein, the third party donor for the non-autologous myeloid progenitor cells is not related to the subject. Additionally or alternatively, in some embodiments of the methods disclosed herein, the third party donor for the non-autologous myeloid progenitor cells is not related to the third party donor for the non-autologous UCB cells.

In any and all embodiments of the methods disclosed herein, the third party donor for the non-autologous UCB cells is not related to the subject and/or is not HLA matched to the subject. Additionally or alternatively, in some embodiments of the methods disclosed herein, the third party donor for the non-autologous myeloid progenitor cells is an adult or a child. The non-autologous myeloid progenitor cells may be isolated from peripheral blood.

In any of the preceding embodiments of the methods disclosed herein, the subject has undergone myeloablation, and optionally has received cyclosporine-A/mycophenolate mofetil to prevent graft versus host disease. The subject may be an adult or a child. Additionally or alternatively, in some embodiments, the non-autologous UCB cells are administered in the absence of antithymocyte globulin (ATG). In certain embodiments, the subject is seropositive for CMV.

Examples of hematologic malignancies or disorders include, but are not limited to, myeloproliferative diseases, lymphomas, myelodysplastic syndrome, amegakaryocytic thrombocytopenia, acute lymphoblastic leukemia, acute myelogenous leukemia, sickle cell disease, beta thalassemia, severe combined immunodeficiency disease, marrow failure, anemia, severe aplastic anemia and Diamond-Blackfan anemia.

In any and all embodiments of the methods disclosed herein, the non-autologous myeloid progenitor cells and the non-autologous UCB cells are HLA matched at 3/8, 4/8, 5/8, 6/8, 7/8, or 8/8 HLA loci, wherein the HLA loci are HLA-A, HLA-B, HLA-C, and HLA-DRB1. In any and all embodiments of the methods disclosed herein, the non-autologous UCB cells and the subject are HLA matched at 4/6, 5/6, or 6/6 HLA loci, wherein the HLA loci are HLA-A, HLA-B, and HLA-DRB1.

In certain embodiments of the methods provided herein, the methods provided herein can be used as a first therapy in combination with one or more second therapies in the treatment of a disorder or condition. Such second therapies include, but are not limited to, surgery, hormone therapy, immunotherapy, phototherapy, or treatment with certain drugs. Exemplary therapies that can be used in combination with the methods provided herein include control of environmental temperature; support with oxygen; a respirator or a ventilator; peripheral blood transfusion; iron supplementation; intravenous feeding; phototherapy; surgery; agents for the treatment of hematologic disorders (including hematologic tumors); antibiotics or antiviral drugs; anti-inflammatory agents (e.g., steroidal anti-inflammatory compounds, non-steroidal anti-inflammatory (NSAID) compounds); nitric oxide; antihistamines; immune suppressants; and immunomodulatory compounds (e.g., a TNF-alpha inhibitor).

### EXAMPLES

The present technology is further illustrated by the following Examples, which should not be construed as limiting in any way.

### Example 1: Experimental Methods

Patients. Patients were treated on a phase II trial (clinicaltrials.gov NCT01682226) between September 2012 and December 2017. The trial was conducted in accordance with the Declaration of Helsinki and was approved by the Memorial Sloan Kettering Cancer Center Institutional Review/Privacy Board. This trial enrolled pediatric and adult patients with high-risk hematologic malignancies without a suitable human leukocyte antigen (HLA) matched related or unrelated donor, who had a suitable umbilical cord blood (CB) graft and a suitable haploidentical donor. For the purposes of this analysis, only adult haplo-dCBT recipients were included to permit comparison with the engraftment kinetics of historic adult dCBT controls. In addition, two patients who underwent identical haplo-dCBT under Single Patient Use were included (one severe aplastic anemia, one whose insurance denied clinical trial participation). All patients were assayed for HLA antibodies as previously described in Dahi PB, et al., Biol Blood Marrow Transplant 20: 735-9 (2014). Antibody titers with mean fluorescence intensity >1000 were considered positive.

CB graft selection. Unit selection was based on unit quality/bank of origin, total nucleated cell (TNC) dose, and donor-recipient HLA-match. Units contained a minimum cryopreserved TNC dose of 1.5×10⁷/kg and were ≥4/6 HLA-A, -B antigen, -DRB1 allele matched to the recipient. Cryopreserved CD34+ cell dose and 8-allele HLA-match were also considered in CB graft selection. The presence of donor-specific HLA antibodies (DSA) against one or both CB units was not a contraindication to unit selection. The HLA-match of the units to each other or the haploidentical donor was not considered.

Haploidentical donor selection and collection. Haploidentical grafts were derived from mobilized peripheral blood; bone marrow harvests were not permitted even in the setting of poor mobilization. Younger adult donors were given priority with emphasis upon availability, compliance, avoidance of a large donor-recipient weight discrepancy, and adequacy of peripheral access. Donors against whom the recipient had DSA were avoided in the latter phase of the trial.

Donors were mobilized with 10 mcg/kg of granulocyte colony stimulating factor (G-CSF) rounded to vial size subcutaneously daily for 5 days. Initially only one collection was performed. The study was later amended to allow a second leukapheresis if the first yielded <3×10⁶/kg CD34+ cells (before CD34+ selection). Grafts were CD34+ cell selected using the CliniMACS CD34 Reagent System (Miltenyi Biotech, Gladbach, Germany) under an Investigational New Device from the US Food and Drug Administration. To guard against permanent haploidentical donor engraftment, the goal for the maximum haploidentical graft CD3+ cell dose was 8×10³/kg. Initially the haplo-CD34+ cell dose was capped at 3×10⁶/kg. Subsequently, the target CD34+ cell dose was increased to ˜5×10⁶/kg without an upper limit.

Conditioning regimens, immunoprophylaxis, and growth factor support. Patients received myeloablative conditioning as described in Barker J N, et al., Blood 105: 1343-1347 (2005) and Ponce D M, et al., Biol Blood Marrow Transplant 19:799-803 (2013). The intensity was based on diagnosis, disease status, age, and hematopoietic cell transplant co-morbidity index (HCT-CI; see Sorror M L, et al. Blood 106: 2912-2919 (2005)) score. High dose conditioning (cyclophosphamide (Cy) 120 mg/kg, fludarabine (Flu) 75 mg/m2, and total body irradiation (TBI) 1375 cGy (Cy 120/Flu 75/TBI 1375)) was considered for fit patients <30 years with hematologic malignancies. Remaining patients received intermediate intensity conditioning (Cy 50 mg/kg, Flu 150 mg/m2, thiotepa (Thio) 10 mg/kg, TBI 400 cGy (Cy 50/Flu 150/Thio 10/TBI 400)) with a reduced Thio dose (5 mg/kg) in patients 60-70 years or those with HCT-CI score ≥5.

CSA and MMF (15 mg/kg every 8 h) were started intravenously on day −3 for graft-versus-host disease prophylaxis. No patient received ATG. ATG was not used in the methods disclosed herein due to its adverse impact on immune reconstitution (Brunstein C G et al., Blood. 2006; 108:2874-80; Komanduri K V et al., Blood. 2007; 110:4543-51; Jacobson C A et al., Biol Blood Marrow Transplant. 2012; 18:565-74; Jain N et al., Leuk Lymphoma. 2013; 54:1242-9; Lindemans C A et al. Blood. 2014; 123:126-32; Admiraal R et al., Blood. 2016; 128:2734-41; Castillo N et al., Biol Blood Marrow Transplant. 2017; 23:491-7) and the substantial evidence of increased mortality in ATG-based CBT (Pascal L et al., Bone Marrow Transplant. 2015; 50:45-50; Pascal L et al., Blood. 2015; 126:1027-32; Shouval R et al., Clin Cancer Res. 2017; 23:6478-86; Tozatto-Maio K et al. Biol Blood Marrow Transplant. 2018; 24:1657-63; Wakamatsu M et al., Blood Adv. 2019; 3:105-15; Ballen K et al., Biol Blood Marrow Transplant. 2020; 26:745-57). All patients received G-CSF 5 mcg/kg/day from day 7 posttransplant until neutrophil recovery. In patients with a second neutrophil nadir, G-CSF was resumed until sustained engraftment was achieved.

Engraftment monitoring and definitions. A white cell count (WCC) differential was obtained once the WCC was >0.5×10⁹/L. Neutrophil recovery was defined as the first of three consecutive days of neutrophils ≥0.5×10⁹/L. Platelet recovery was the first day of ≥20×10⁹/L platelets without transfusion for 7 consecutive days. Graft failure was defined as requirement for a second stem cell infusion or death without neutrophil recovery on day 28 or later.

Haploidentical and CB donor chimerism were monitored using PCR amplification of informative recipient and donor short tandem repeats. Whole blood assays were done on days 14, 28, 60, 100, 180, and 365 posttransplant. White cell subset chimerism analyses were performed in sorted myeloid, T-, B- and NK-cell subsets (purity >95%) on days 28, 100, and 365 posttransplant. Analysis of lineage-specific chimerism was foregone if the purity threshold was not achieved or if the specific cell subset count was too low. Of the two CB units infused, the dominant (or engrafting) CB unit was the only one detected or the one with sustained >50% contribution to CB-derived chimerism.

Statistical methods. The objective was to determine the speed and success of sustained myeloid recovery after haplo-dCBT. Success was arbitrarily defined as neutrophil recovery by 2 weeks posttransplant (prior to or on day 14). Cumulative incidences of neutrophil and platelet recovery were estimated considering early death as a competing risk. Chimerism was analyzed using summary statistics and box-and-whisker diagrams. Correlation of haplo-CD34+ and CB cell doses with days to neutrophil recovery was evaluated using Spearman's rank correlation coefficient. Cell doses of dominant and non-dominant CB units were compared using the Wilcoxon signed-rank test. Univariate and multivariate logistic regression analyses were performed in patients who achieved sustained CB engraftment to evaluate factors associated with higher odds of successful haplo-CD34+ myeloid bridging. All variables with p<0.10 in univariate analysis were included in the multivariate model. Transplant-related mortality (TRM) was compared across WCC recovery groups using Gray's test in a day 28 landmark analysis considering relapse as a competing risk. Results with two-tailedp values <0.05 were considered significant. All analyses were conducted using R statistical software, version 3.1.1 (R Foundation for Statistical Computing, Vienna, Austria).

### Example 2: Patient and Graft Characteristics

Seventy-eight patients (median age 48 years (range 21-68), median weight 82 kg (range 48-138)) underwent haplod-CBT. Thirty-seven patients (47%) were male and 44 (56%) were CMV seropositive. Diagnoses included 54 (69%) acute leukemias, 10 (13%) myelodysplasia/myeloproliferative diseases, 13 (17%) lymphomas, and 1 aplastic anemia. Three patients were second allograft recipients. Conditioning was high dose (Cy 120/Flu 75/TBI 1375, n=1) or intermediate intensity (Cy 50/Flu 150/Thio 10/TBI 400 (n=64), Cy 50/Flu 150/Thio 5/TBI 400 (n=13)).

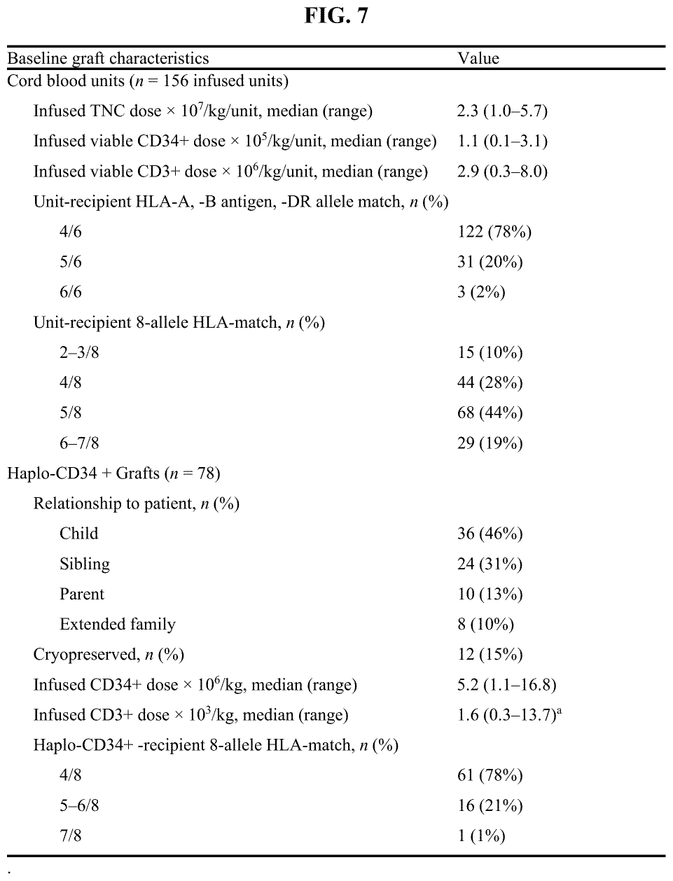

Infused CB unit (n=156) and haplo-CD34+ (n=78) graft characteristics are shown in FIG. 7. CB units had a median infused TNC dose of 2.3 (range 1.0-5.7)×10⁷/kg/unit and a median infused viable CD34+ cell dose of 1.1 (range 0.1-3.1)×10⁵/kg/unit. The median infused viable CD3+ cell dose was 2.9 (range 0.3-8.0)×10⁶/unit. The majority of units were 4/6 HLA-A, -B antigen, -DRB1 allele matched to the patient, and the median CB unitrecipient HLA-allele match was 5/8 (range 2-7/8). Seven patients (9%) had DSA against their CB graft.

Haplo-CD34+ grafts were most commonly procured from children (46%) or siblings (31%). Haploidentical donors had a median age of 33 years (range 15-71). The median infused CD34+ dose was 5.2 (range 1.1-16.8)×10⁶/kg. The median infused CD3+ cell dose was 1.6 (range 0.3-13.7)×10³/kg and approximately three logs lower than that of the CB units. The majority of haplo-CD34+ grafts (n=61, 78%) were 4/8 HLA-allele matched to the patient. Eleven patients (14%) had DSA against their haploidentical graft.

### Example 3: Overall Hematopoietic Engraftment

Of the 78 analyzed patients, 75 engrafted, 2 had graft failure, and 1 heavily pretreated patient died on day 14 from veno-occlusive disease and multi-organ failure. The cumulative incidence of sustained neutrophil recovery for the entire cohort was 96% (95% CI: 87-99). The day 100 cumulative incidence of platelet recovery was 87% (95% CI: 77-93).

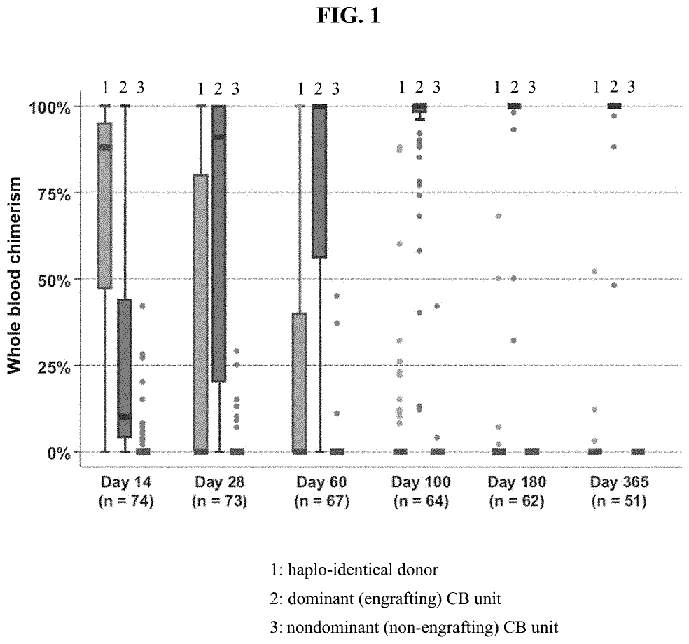

In the 75 engrafting patients, sustained engraftment was mediated by a dominant CB unit. The dominant units had a median infused viable CD34+ cell dose of 1.23 (range 0.24-2.95)×10⁵/kg and a median infused viable CD3+ cell dose of 1.02 (range 0.14-3.4)×10⁶/kg. The median HLA-match of the dominant CB unit to the recipient and to the haploidentical graft were 5/8 (range 3-7/8) and 3/8 (range 1-7/8), respectively. The pattern of whole blood chimerism is shown in FIG. 1. Overall, haploidentical grafts predominated early posttransplant (median day 14 chimerism 88% (range 0-100)). However, no haploidentical graft was detected in 51% of evaluable patients at day 28, in 61% at day 60, and in 78% at day 100 posttransplant. In addition, the haploidentical graft comprised only a minor component of donor chimerism in nearly all remaining patients. Concurrently, the dominant CB unit whole blood chimerism increased from a median of 10% (range 0-100) at day 14 to 91% (range 0-100) at day 28, 100% (range 0-100) at day 60, and 100% (range 12-100) at day 100 and beyond. White cell subset chimerism analysis revealed that loss of the haploidentical graft was associated with early dominant CB unit chimerism in the T-cell fraction.

At a median survivor follow-up of 3 years and 9 months (range 1-6 years) all evaluable patients maintain engraftment with a dominant CB unit.

### Example 4: Patterns of Hematopoietic Recovery

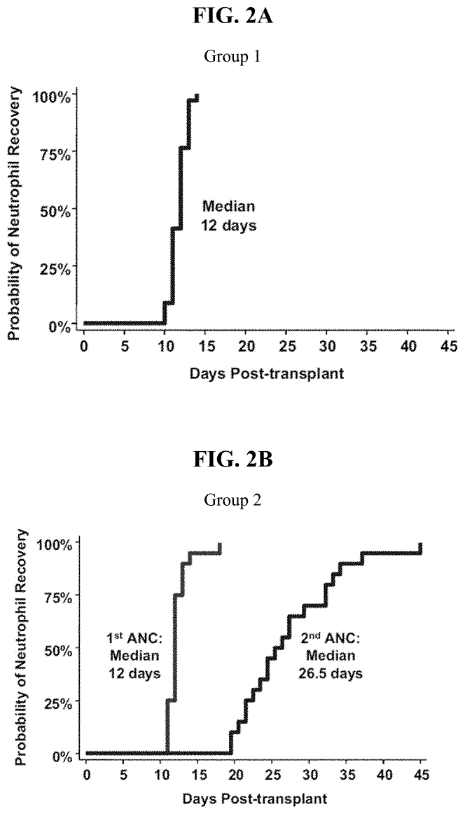

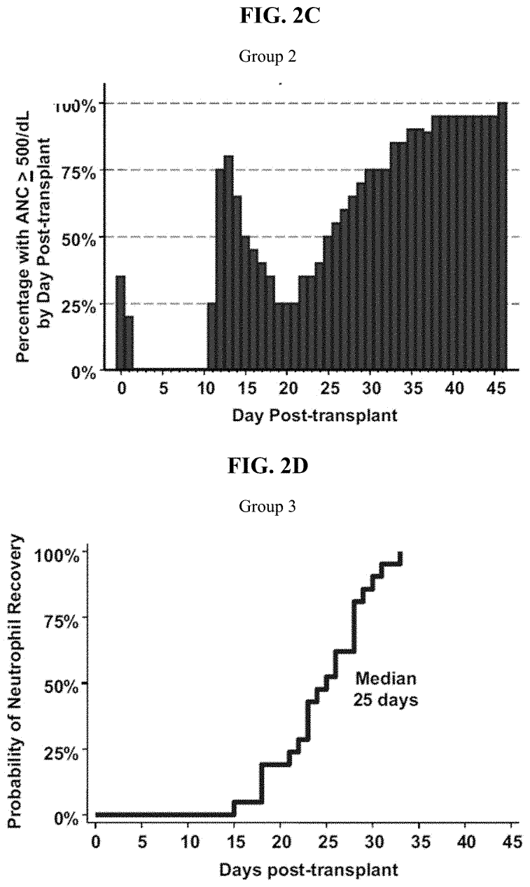

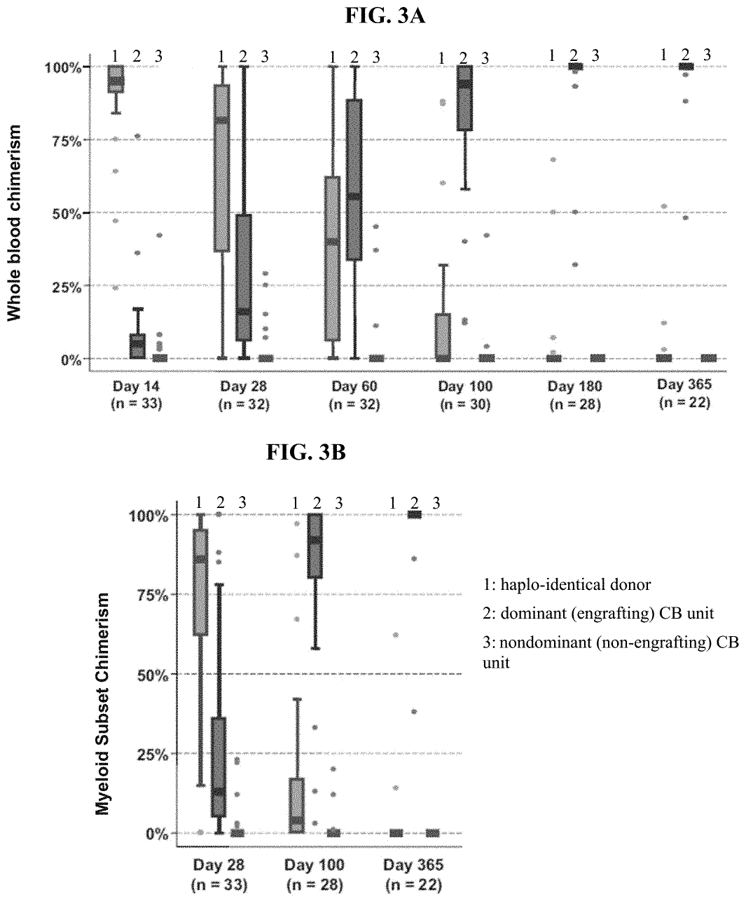

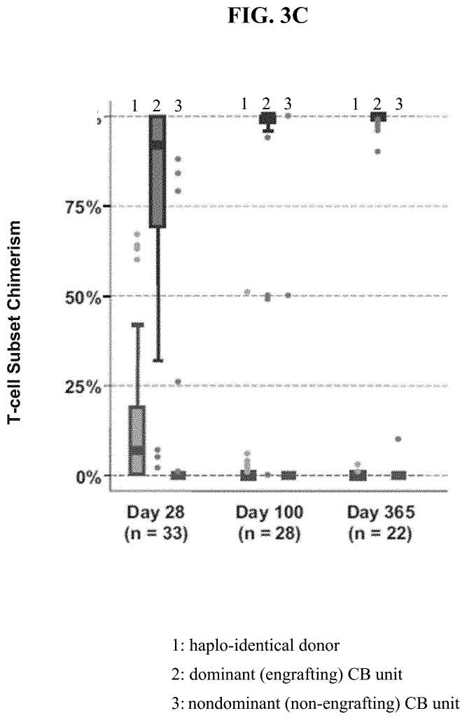

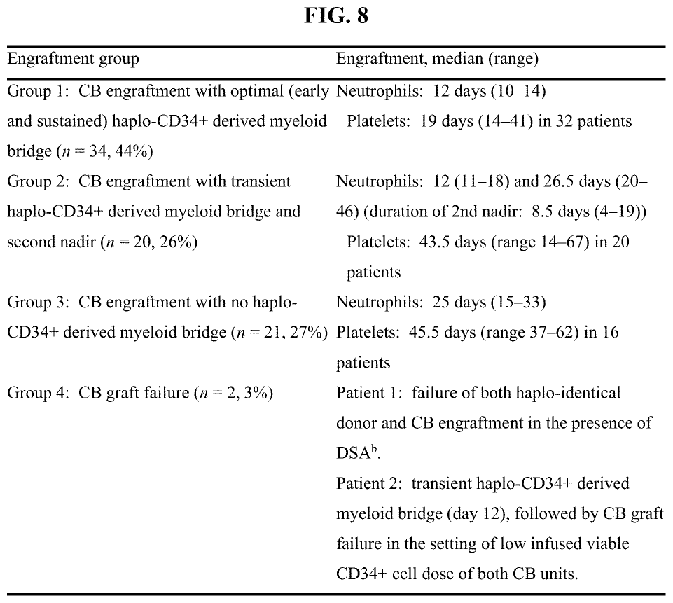

While 75 of 77 evaluable patients had sustained CB engraftment that was mediated by a dominant CB unit, the success of obtaining an early haploidentical donor-derived myeloid bridge was variable between patients. Three distinct engraftment patterns were observed (FIG. 8, FIGS. 2A-2D) and were characterized by distinct chimerism patterns associated with the speed of rejection of the haplo-CD34+ graft by the dominant CB unit (FIGS. 3A-3C, 4A-4C, and 5A-5C).

Group 1 patients (34/77, 44%) had early sustained myeloid recovery at a median of 12 days (range 10-14) posttransplant (FIG. 2A). This rapid recovery was almost always mediated by the haploidentical graft with subsequent transition to CB-derived hematopoiesis. These patients had a high median day 14 haploidentical graft whole blood chimerism of 95% (range 24-100) (FIG. 3A). Subsequently, they had increasing dominant CB unit chimerism. By day 180, the haploidentical graft was detected in only a minority (5/28, 18%) of patients (median haploidentical donor chimerism 7%, range 2-68). By 1 year, haploidentical cells were only detected in three patients (contributions 3%, 12% and 52%) with two having since converted to 100% dominant CB unit. In white cell subset analyses, the majority of myeloid cells were derived from the haploidentical donor at day 28 (FIG. 3B). In contrast, T cells were derived from the dominant CB unit (FIG. 3C) with a progressive increase in dominant CB unit derived chimerism in all lineages thereafter. In Group 1 patients, a higher infused haplo-CD34+ dose was associated with faster neutrophil recovery (r=−0.74, p<0.001). Platelet recovery was also enhanced in these patients (FIG. 8).

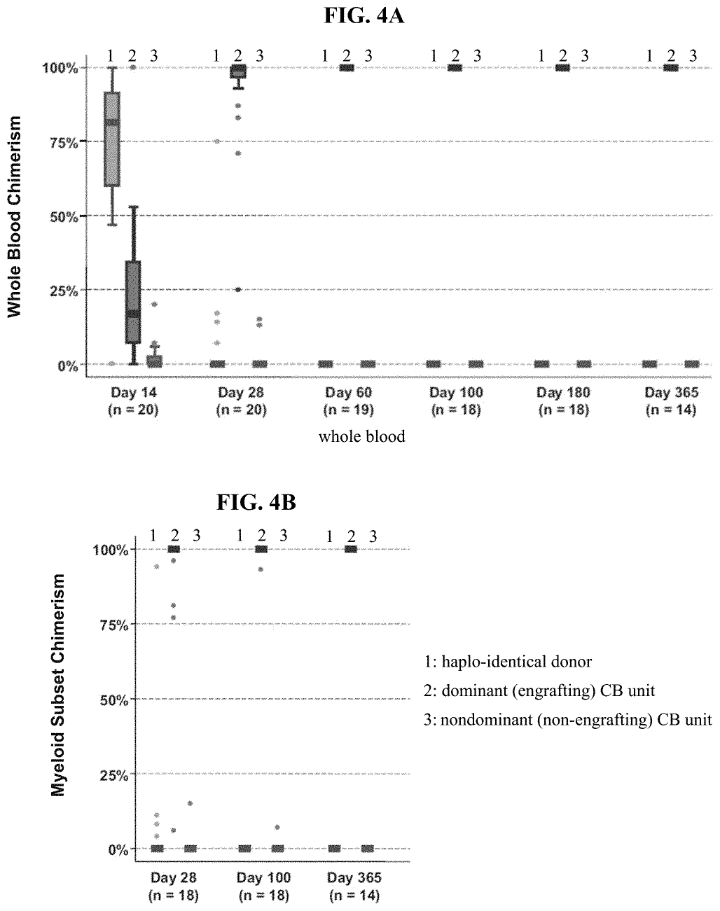

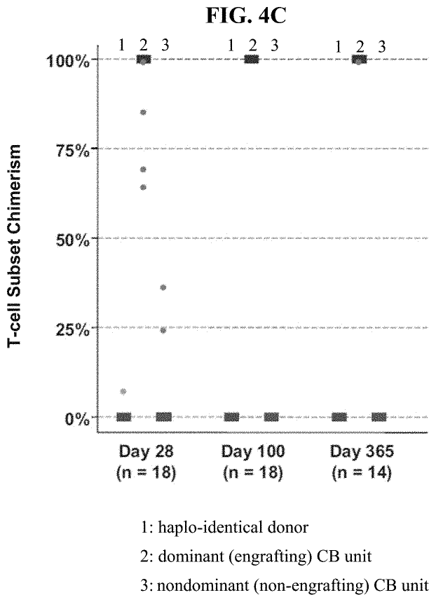

Group 2 patients (20/77, 26%) had initial myeloid recovery (defined as a neutrophil count ≥0.5 k/mcL for ≥1 day) followed by a second nadir (<0.5 k/mcL for ≥2 consecutive days) preceding sustained engraftment (FIGS. 2B-2C). The median times to first and second neutrophil recoveries were 12 days (range 11-18) and 26.5 days (range 20-46), respectively. Short-lived haplo-CD34+ engraftment accounted for the transient myeloid bridge (median day 14 haploidentical donor chimerism 82% (range 0-100)), although in two patients the haploidentical donor was undetectable by 2 weeks posttransplant (FIG. 4A). This was followed by sustained CB-derived hematopoiesis (median whole blood chimerism 100% dominant CB unit by day 60 and beyond). In addition, myeloid and T-cell lineages were primarily derived from the dominant CB unit as early as day 28 (FIGS. 4B-4C). Notably, a higher dominant CB unit infused viable CD3+ dose correlated with a slower time to the first (haploidentical donor-derived) neutrophil recovery (r=0.43, p=0.058), whereas a higher dominant CB unit infused viable CD34+ cell dose correlated with a faster time to the second (CB-derived) neutrophil recovery (r=−0.44, p=0.052), although statistical significance at the 0.05 level was not reached.

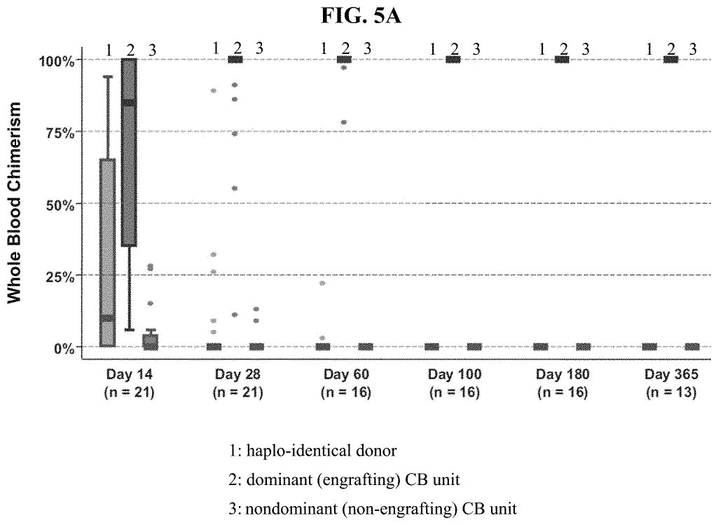

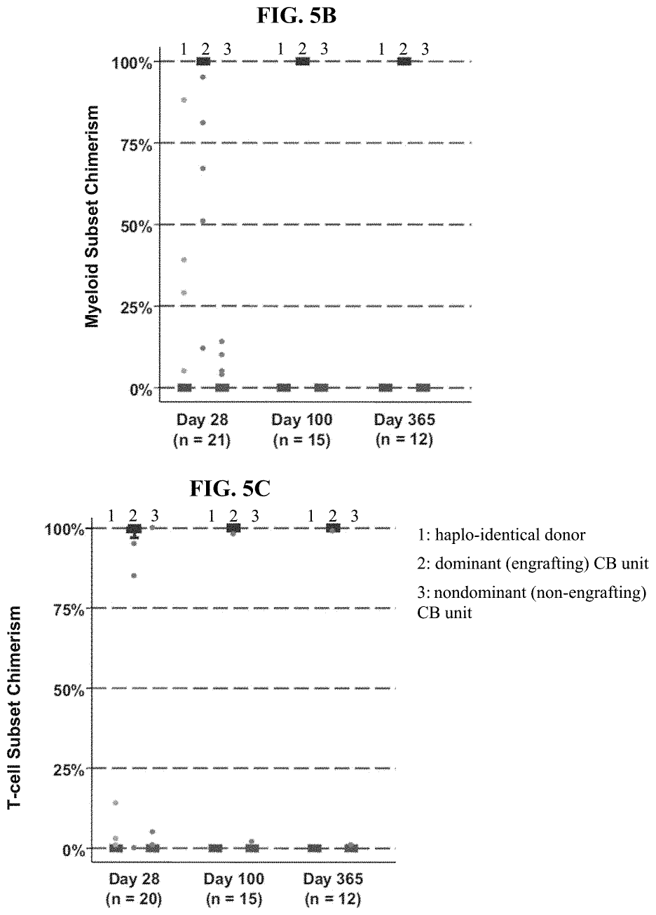

Group 3 patients (21/77, 27%) had delayed neutrophil recovery (median 25 days, range 15-33) (FIG. 2D). At day 14 posttransplant, the majority had either no or minimal haploidentical donor chimerism (median 10% (range 0-94)) in whole blood (FIG. 5A), and no haploidentical donor was ever detected in nine patients. In addition, seven patients had no myeloid bridge despite a day 14 haploidentical donor contribution >50%. By day 60, all but two patients were 100% donor with the dominant CB unit, and this unit accounted for hematopoiesis in all patients subsequently. Moreover, in subset analysis, the dominant CB unit was either the only or greatest contributor to all lineages as of day 28 and beyond (FIGS. 5B-5C). In Group 3 patients, a higher infused viable CD34+ cell dose of the dominant CB unit was associated with faster neutrophil recovery, although this correlation was not significant at the 0.05 level (r=−0.42, p=0.06).

The two remaining evaluable patients (Group 4, 3%) had graft failure (FIG. 8). One, a prior allograft recipient, with DSA against the haploidentical donor and both CB units had failure of haplo-CD34+ and CB engraftment. The second had transient haploidentical donor-derived neutrophil recovery followed by failure of CB engraftment. CB graft failure was likely due to the very low infused viable CD34+ cell doses of each unit (0.22×105/kg and 0.35×105/kg, respectively). Both patients were successfully re-transplanted with single CB units.

### Example 5: Factors Associated with an Optimal Myeloid Bridge

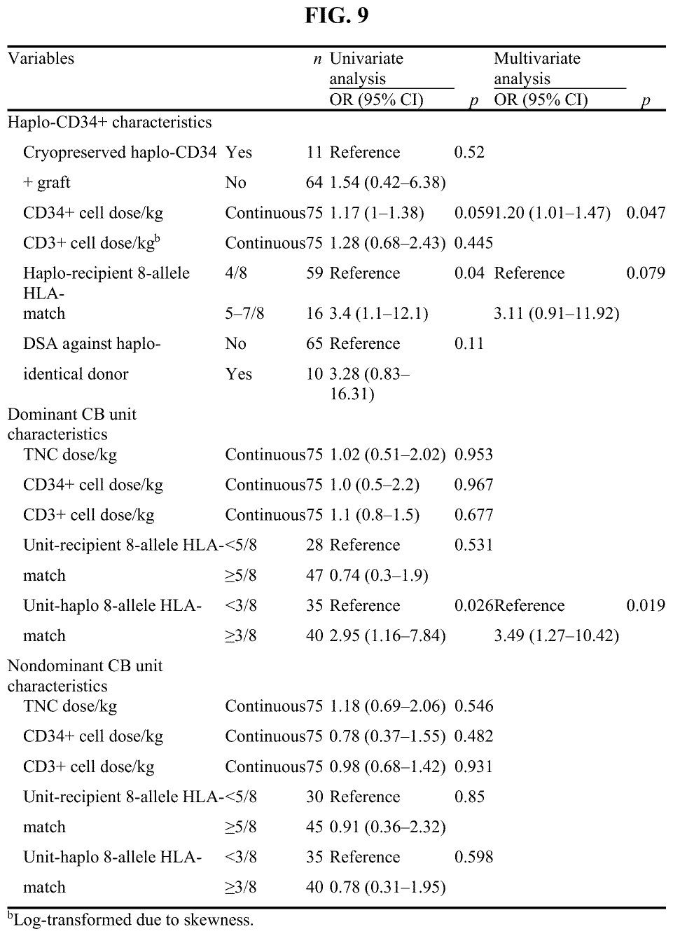

Next, the association between graft characteristics and the likelihood of achieving an optimal myeloid bridge was investigated (FIG. 9). This was defined as early, haploidentical donor-derived, sustained neutrophil recovery by 2 weeks posttransplant (i.e., without a second nadir) prior to CB engraftment. In multivariate analysis, a higher haplo-CD34+ dose (OR: 1.2 (95% CI: 1.01-1.47), p=0.047) significantly improved the odds of achieving an optimal myeloid bridge. While there was no haploidentical CD34+dose threshold that could guarantee optimal bridging, none of the eight patients who received a haplo-CD34+ cell dose <3×106/kg had a bridge. Furthermore, a ≥3/8 HLA-match of the dominant CB unit to the haploidentical donor was also associated with higher odds of optimal bridging (OR: 3.49 (95% CI: 1.27-10.42), p=0.019).

A ≥5/8 HLA-match of the haploidentical donor to the recipient was associated with optimal myeloid bridging in the univariate but not the multivariate model. Presence of DSA against the haploidentical graft had no impact. Cell dose and HLA-match of the non-dominant CB unit were also not associated with the likelihood of optimal bridging.

### Example 6: Association of Optimal Myeloid Bridging with Duration of Hospitalization and Day 100 TRM

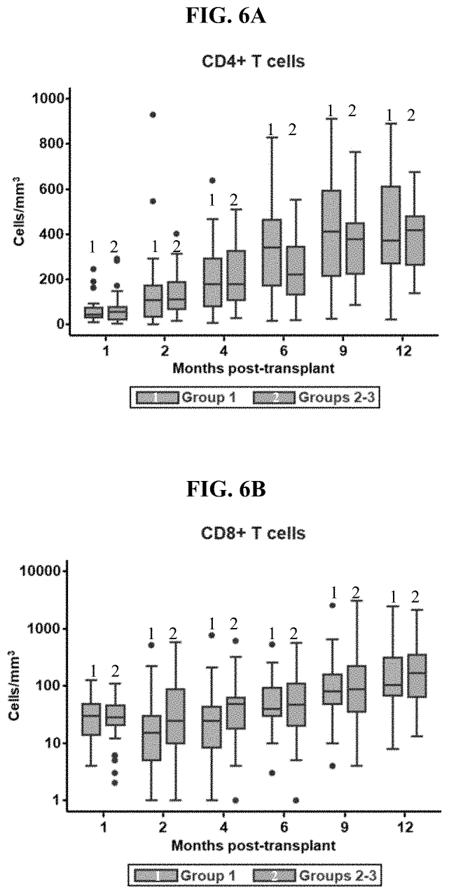

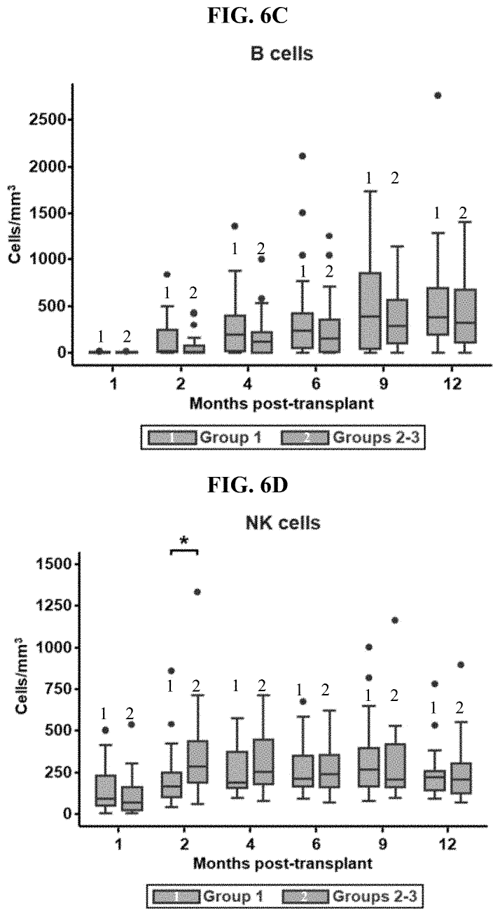

Of 70 engrafted patients discharged from their initial hospitalization, Group 1 patients with sustained myeloid bridge were discharged earlier (median 28 days (range 20-60)) than the Group 2-3 patients with transient or no bridging (median 36 days (range 28-98)). However, the day 100 TRM in Group 1 patients was not different than that of Group 2-3 patients (9% (95% CI: 2-21) versus 15% (95% CI: 6-27), p=0.388). In addition, optimal bridging in Group 1 patients was not associated with improved immune recovery (FIGS. 6A-6D).

### Example 7: Determinants of CB Unit Dominance

CB unit dominance was associated with a higher infused viable CD3+ cell dose (median dose 3.3 (range: 0.9-8.0)×106/kg versus median 2.6 (range: 0.3-6.0)×106/kg, p<0.001). In contrast, CB infused TNC dose, infused viable CD34+ cell dose and 8-allele CB unit-recipient HLA-match were not significant (data not shown).

### Example 8: Myeloid Bridging CBT Patients that Receive Unrelated Third-Party Donor Myeloid Progenitor Cells

A new clinical trial will be developed in which third-party myeloid progenitors will be co-transplanted with a single CB unit, but in this trial the third-party donor will be chosen to match the CB graft but not the recipient patient. This planned trial that aims to achieve an early myeloid bridge derived from the third party cells will utilize a CD34+ selected unrelated volunteer donor graft. Alternatively, a similar approach could be done with third-party cells obtained from an expanded CB unit selected to match an unmanipulated CB graft. It is anticipated that patients that receive third-party donor myeloid progenitors that are HLA-matched to the CB graft, but not the recipient patient per se, will show improvements in myeloid bridging that are comparable or greater than those observed in patients that receive haploidentical myeloid progenitors that are HLA-matched to the CB graft.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating a hematologic malignancy or disorder in a subject in need thereof comprising
administering to the subject an effective amount of non-autologous myeloid progenitor cells, and an effective amount of non-autologous umbilical cord blood (UCB) cells,
wherein the non-autologous UCB cells and the non-autologous myeloid progenitor cells are HLA matched and
wherein the non-autologous myeloid progenitor cells and the non-autologous UCB cells are obtained from different third party donors.

2. A method for enhancing early myeloid recovery in a subject that is receiving umbilical cord blood (CB) transplantation comprising
administering to the subject an effective amount of non-autologous myeloid progenitor cells, and an effective amount of non-autologous umbilical cord blood (CB) cells,
wherein the non-autologous UCB cells and the non-autologous myeloid progenitor cells are HLA matched and
wherein the non-autologous myeloid progenitor cells and the non-autologous UCB cells are obtained from different third party donors, optionally wherein the subject is suffering from a hematologic malignancy or disorder.

3. The method of claim 1, wherein the non-autologous myeloid progenitor cells and/or non-autologous UCB cells express CD34.

4. The method of claim 1, wherein the non-autologous UCB cells are administered as a single unit or multiple units and/or wherein the UCB cells have been cryopreserved.

5. The method of claim 2, wherein myeloid recovery comprises recovery of one or more of granulocytes, basophils, eosinophils, neutrophils, megakaryocytes, platelets, erythrocytes, monocytes, and macrophages.

6. The method of claim 1, wherein the non-autologous UCB cells are administered at a total nucleated cell (TNC) dose of about 1.0×10⁷/kg/unit to about 5.7×10⁷/kg/unit.

7. The method of claim 1, wherein the non-autologous myeloid progenitor cells are administered at a dose of about 0.1×10⁵/kg/unit −3.1×10⁵/kg/unit.

8. The method of claim 1, wherein the third party donor for the non-autologous myeloid progenitor cells is haploidentical to the subject and/or wherein the third party donor for the non-autologous myeloid progenitor cells is not related to the subject.

9. The method of claim 1, wherein the third party donor for the non-autologous myeloid progenitor cells is not related to the third party donor for the non-autologous UCB cells.

10. The method of claim 1, wherein the third party donor for the non-autologous UCB cells is not related to the subject and/or is not HLA matched to the subject.

11. The method of claim 1, wherein the subject is an adult or a child.

12. The method of claim 1, wherein the third party donor for the non-autologous myeloid progenitor cells is an adult or a child.

13. The method of claim 1, wherein the non-autologous myeloid progenitor cells are isolated from peripheral blood.

14. The method of claim 1, wherein the subject has undergone myeloablation and optionally has received cyclosporine-A/mycophenolate mofetil to prevent graft versus host disease.

15. The method of claim 1, wherein the non-autologous UCB cells are administered in the absence of antithymocyte globulin (ATG).

16. The method of claim 1, wherein the hematologic malignancy or disorder is selected from the group consisting of myeloproliferative diseases, lymphomas, myelodysplastic syndrome, amegakaryocytic thrombocytopenia, acute lymphoblastic leukemia, acute myelogenous leukemia, sickle cell disease, beta thalassemia, severe combined immunodeficiency disease, marrow failure, anemia, severe aplastic anemia and Diamond-Blackfan anemia.

17. The method of claim 1, wherein the subject is seropositive for CMV.

18. The method of claim 1, wherein the non-autologous myeloid progenitor cells are administered separately, sequentially, or simultaneously with non-autologous UCB cells.

19. The method of claim 1, wherein the non-autologous myeloid progenitor cells and the non-autologous UCB cells are HLA matched at 3/8, 4/8, 5/8, 6/8, 7/8, or 8/8 HLA loci, wherein the HLA loci are HLA-A, HLA-B, HLA-C, and HLA-DRB1.

20. The method of claim 1, wherein the non-autologous UCB cells and the subject are HLA matched at 4/6, 5/6, or 6/6 HLA loci, wherein the HLA loci are HLA-A, HLA-B, and HLA-DRB1.
